(19) United States
(12) Patent Application Publication
(10) Pub. No.: US 2016/0209554 A1
(43) Pub. Date: Jul. 21, 2016

(54) Fabrication Method of Lens Substrate Having Anti-Fog Film Layer and Structure Thereof

(71) Applicant: Sunever Eyewear Co., Ltd., Tainan City (TW)

(72) Inventor: Yan-Huang Cheng, Tainan City (TW)

(21) Appl. No.: 14/599,571

(22) Filed: Jan. 19, 2015

(51) Int. Cl. G02B 1/18; C25D 7/00; A61F 9/02

(52) U.S. Cl. CPC G02B 1/18; A61F 9/029; C25D 7/00

(57) ABSTRACT

A fabrication method of a lens substrate having an anti-fog film layer and a structure thereof, where the lens substrate comprises an anti-fog film layer formed with a predetermined configuration and a proper thickness (preferably substantially between 0.1 mm and 0.3 mm) by cellulose acetate, and a lens (i.e., a substrate layer) formed by electroplating and combining with the anti-fog film layer to form the lens substrate, wherein an overall thickness of the lens substrate is substantially thinned, and predetermined anti-fog properties of the lens substrate are kept after repeated cleaning or wiping after long terms.

### BACKGROUND OF THE INVENTION

1. Technical Field

The invention relates to a fabrication method of a lens substrate having an anti-fog film layer and a structure thereof, and more particularly, to a lens substrate comprising an anti-fog film layer formed with a predetermined configuration and a proper thickness (substantially between 0.1 mm and 0.3 mm) by cellulose acetate, and a lens (i.e., a substrate layer) formed by electroplating and combining with the anti-fog film layer to form the lens substrate, wherein an overall thickness of the lens substrate is substantially thinned, and predetermined anti-fog properties of the lens substrate are kept after repeated cleaning or wiping after long terms.

2. Description of Related Art

For goggles or similar glasses that protect parts of the human face and eyes such as glasses or goggles used in skiing, motor-cross racing, biking, car driving, sports, and safety operations in industrial environment. Anti-fog films are combined on the lenses of such glasses to provide anti-fog properties when being worn, so as to protect parts of the human face and eyes and provide expected anti-fog properties during usage. There are many implementation types to provide such glasses with anti-fog properties, and the ultimate goal of which is to allow the lens substrate to have structures with safety and anti-fog applications.

A first conventional type that allows the lens substrate to have an anti-fog film layer is disclosed here (as shown in FIG. 1); the type includes immersing a lens 10 in a chemical agent (i.e., an anti-fog agent) for a predetermined time (or may be substituted by coating), and carrying out electroplating processes on the lens 10 after the immersion to firmly cover an anti-fog film 20 in the above on the lens 10. If superior anti-fog properties are desired for the lens substrate, the time of immersion should be extended so that the applied anti-fog agent may adhere better and the surface of the lens substrate may be thicker; after processing the above anti-fog coating, subsequent electroplating processes may result in bubbles on the surface of the lens substrate and cause the fraction of defective lens substrates to increase.

The above implementation type that allows the lens substrate to have an anti-fog film layer has the following disadvantages:

1. If superior anti-fog properties are desired for the lens substrate, satisfactory electroplating processing may not be simultaneously achieved, unless the lens substrate only undergoes mere anti-fog agent application or mere electroplating coating; however, such practices result in complication of the processing, and relatively increase cost and reduce production speed, which are not acceptable for most customers.

2. Such anti-fog coating adheres on the lens substrate by immersion, and the anti-fog properties of the subsequently cut lenses may gradually diminish as the wearer wipes the lenses, and thus the expected anti-fog properties of the lens surface may be lost.

A second conventional type that allows the lens substrate to have an anti-fog film layer is also disclosed here (as shown in FIG. 2); in this type, each lens 30 includes a basic lens 301 and an anti-fog lens 302, wherein a planar component with a predetermined configuration and a proper thickness is immersed in a chemical agent (i.e., an anti-fog agent) for a predetermined time for anti-fog processing, and the anti-fog lens 302 is formed (preferably substantially between 0.4 mm and 1.0 mm). A lens with a predetermined configuration and a proper thickness is subsequently processed by electroplating to form the basic lens 301; subsequently, the anti-fog lens 302 and the basic lens 301 use a peripheral foam 303 with a predetermined thickness and same periphery as a connection medium, wherein adhesives 304 adhere the two components (i.e., the anti-fog lens 302 and the basic lens 301) on the two sides of the peripheral foam 303, respectively, to form the lens 30. However, the curvatures of the two lenses (i.e., the anti-fog lens 302 and the basic lens 301) cannot be exactly the same, and thus the formed lens may easily cause the user to suffer from vertigo when wearing.

The above implementation type that allows the lens substrate to have an anti-fog film layer has the following disadvantages:

1. Since the lens substrate includes a basic lens and an anti-fog lens using a peripheral foam as a connection medium, the product thickness of the formed lens substrate is rather large.

2. After terms of usage, the foam between the basic lens and the anti-fog lens of the lens substrate may cause water to leak into between the two lenses from the outside as the user repeatedly washes the lens.

3. The anti-fog coating on a side of the anti-fog lens of the lens substrate adheres on the predetermined lens by immersion, and the anti-fog properties of the subsequently cut lenses may gradually diminish as the wearer wipes the lenses, and thus the expected anti-fog properties of the lens surface may be lost.

The present invention seeks to substantially overcome the disadvantages of the implementation of the above similar conventional types that form lens substrates to have anti-fog film layers, so that a lens substrate comprises an anti-fog film layer formed with a predetermined configuration and a proper thickness (preferably substantially between 0.1 mm and 0.3 mm) by cellulose acetate, and a lens (i.e., a substrate layer) formed by electroplating and combining with the anti-fog film layer to form the lens substrate, wherein an overall thickness of the lens substrate is substantially thinned, and the anti-fog film layer on a side of the lens substrate has permanent anti-fog properties so that predetermined anti-fog properties of the lens substrate are kept after repeated cleaning or wiping after long terms.

### BRIEF SUMMARY OF THE INVENTION

The present invention seeks to further improve the disadvantages of the implementation and usage of the conventional lens substrates formed to have anti-fog film layers, so that a lens substrate comprises an anti-fog film layer formed with a predetermined configuration and a proper thickness (substantially between 0.1 mm and 0.3 mm) by cellulose acetate, and a lens (i.e., a substrate layer) formed by electroplating and combining with the anti-fog film layer to form the lens substrate, wherein an overall thickness of the lens substrate is substantially thinned, and predetermined anti-fog properties of the lens substrate are kept after repeated cleaning or wiping after long terms.

A first purpose of the present invention is to provide a fabrication method of a lens substrate having an anti-fog film layer, wherein the lens substrate is formed to achieve substantial thinning and keep expected anti-fog properties, the fabrication method comprising: forming the anti-fog film layer with a predetermined configuration and a proper thickness (preferably substantially between 0.1 mm and 0.3 mm) by cellulose acetate, wherein the lens substrate comprises the anti-fog film layer and a substrate layer; forming the substrate layer by electroplating a component with a proper thickness that is transparent or not completely transparent; and combining the anti-fog film layer and the substrate layer by an adhesive or non-adhesive to form the lens substrate.

A second purpose of the present invention is to provide a structure of a lens substrate having an anti-fog film layer, wherein the lens substrate is formed to achieve substantial thinning and keep expected anti-fog properties, the structure comprising: an anti-fog film layer, formed with a predetermined configuration and a proper thickness by cellulose acetate; and a substrate layer, formed by electroplating, combined on a lower surface of the anti-fog film layer by an adhesive or non-adhesive to form the lens substrate, wherein an overall thickness of the lens substrate is substantially thinned, and predetermined anti-fog properties of the lens substrate are kept after repeated cleaning or wiping after long terms.

A third purpose of the present invention is that the proper thickness of the anti-fog film layer of the lens substrate is substantially between 0.1 mm and 0.3 mm.

A fourth purpose of the present invention is that the adhesive combining the anti-fog film layer and the substrate layer of the lens substrate is implemented by an optically clear adhesive (OCA), ultraviolet (UV) curable adhesive, optically clear resin (OCR), or highly transparent adhesive.

A fifth purpose of the present invention is that the substrate layer of the lens substrate is implemented by polycarbonate (PC), triacetate cellulose (TAC) film, NYLON, CR-39 resin lens, polymethylmethacrylate (PMMA), polyethylene terephthalate (PET), or polypropylene (PP).

A sixth purpose of the present invention is that the substrate layer of the lens substrate is implemented by a component that is transparent or otherwise colored and is not completely transparent.

A seventh purpose of the present invention is that the substrate layer of the lens substrate is implemented with a spherical shape, a non-spherical shape, a planar shape, or a plate with a predetermined thickness and a configuration with a plurality of protrusions.

An eighth purpose of the present invention is that a surface of the substrate layer of the lens substrate is implemented to combine anti-scratch hard coating, UV400 protection, photochromic coating, or electroplating coatings such as multi-layer coating, water repellent coating, oleophobic coating, antireflection coating, anti-blue-ray coating, mirror coating, and color mirror coating.

A ninth purpose of the present invention is that the anti-fog film layer of the lens substrate is implemented with the predetermined Configuration of a curved surface or non-curved surface according to a configuration of the substrate layer correspondingly.

### BRIEF DESCRIPTION OF THE SEVERAL VIEWS OF THE DRAWINGS

FIG. 1 is a cross-section view of a first conventional lens substrate having an anti-fog film layer.

FIG. 2 is a cross-section view of a second conventional lens substrate having an anti-fog film layer.

FIG. 3 is a cross-section view of a lens substrate having an anti-fog film layer of the invention.

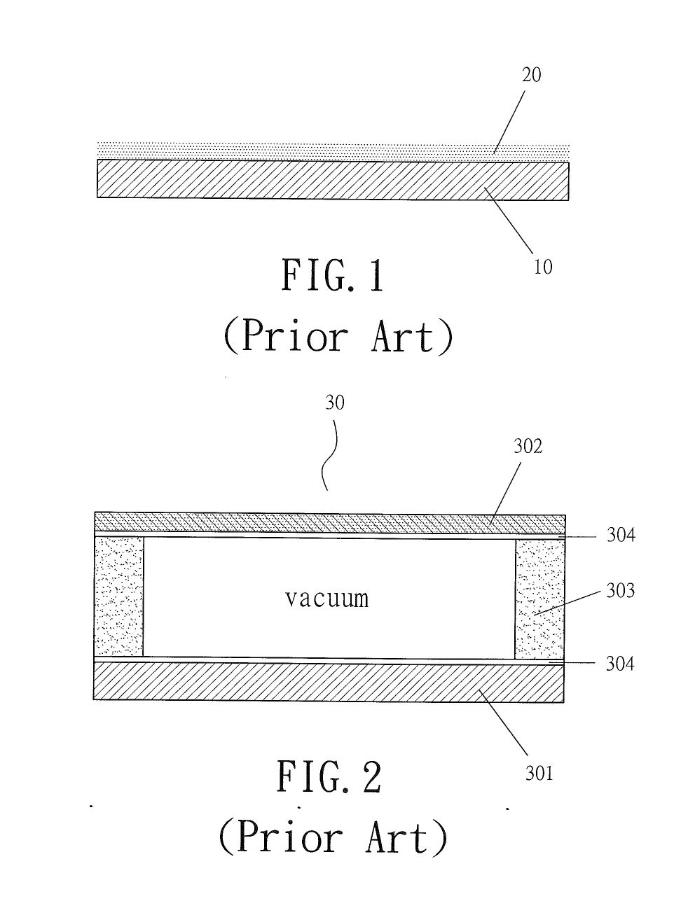

### DETAILED DESCRIPTION OF THE INVENTION

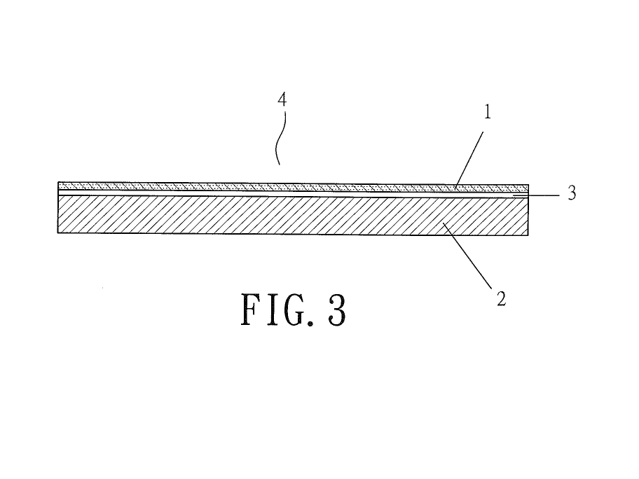

A fabrication method of a lens substrate having an anti-fog film layer designed in the present invention (as shown in FIG. 3), wherein the lens substrate is formed to achieve substantial thinning and keep expected anti-fog properties, comprises: forming the anti-fog film layer 1 with a predetermined configuration and a proper thickness (preferably substantially between 0.1 mm and 0.3 mm) by cellulose acetate, wherein the lens substrate 4 comprises the anti-fog film layer 1 and a substrate layer 2; forming the substrate layer 2 by electroplating a component with a proper thickness that is transparent or not completely transparent; and combining the anti-fog film layer 1 and the substrate layer 2 by an adhesive 3 or non-adhesive to form the lens substrate 4.

A structure of the lens substrate 4, wherein the lens substrate 4 is formed to achieve substantial thinning and keep expected anti-fog properties, comprises (as shown in FIG. 3): an anti-fog film layer 1, formed with a predetermined configuration and a proper thickness (preferably substantially between 0.1 mm and 0.3 mm) by cellulose acetate; and a substrate layer 2 (the substrate layer 2 here is implemented by a component that is transparent or otherwise colored and is not completely transparent), formed by electroplating, combining with the anti-fog film layer 1 by an adhesive S or non-adhesive to form the lens substrate 4.

The adhesive 3 combining the anti-fog film layer 1 and the substrate layer 2 of the lens substrate 4 is implemented by an optically clear adhesive (OCA), ultraviolet (UV) curable adhesive, optically clear resin (OCR), or highly transparent adhesive; the substrate layer 2 of the lens substrate 4 is implemented by polycarbonate (PC), triacetate cellulose (TAC) film, NYLON, CR-39 resin lens, polymethylmethacrylate (PMMA), polyethylene terephthalate (PET), or polypropylene (PP); the substrate layer 2 of the lens substrate 4 is implemented with a spherical shape, a non-spherical shape, a planar shape, or a plate with a predetermined thickness and a configuration with a plurality of protrusions; a surface of the substrate layer 2 of the lens substrate 4 is implemented to combine anti-scratch hard coating, UV400 protection, photochromic coating, or electroplating coatings such as multi-layer coating, water repellent coating, oleophobic coating, antireflection coating, anti-blue-ray coating, mirror coating, and color mirror coating; the anti-fog film layer 1 of the lens substrate 4 is implemented with the predetermined configuration of a curved surface or non-curved surface according to a configuration of the substrate layer correspondingly.

The implementation type of the above lens substrate having an anti-fog film layer has the following advantages:

1. The anti-fog film layer of the lens substrate is a thin film that may directly adhere to a side of the substrate layer, and combines to yield a lens substrate with substantially thinned overall thickness.

2. The anti-fog film layer of the lens substrate is formed by cellulose acetate, and the superior anti-fog properties of the material of the anti-fog film layer ensures that predetermined anti-fog properties of the lens substrate are kept after repeated cleaning or wiping after long terms.

What is claimed is:

1. A fabrication method of a lens substrate having an anti-fog film layer, wherein the lens substrate is formed to achieve substantial thinning and keep expected anti-fog properties, the fabrication method comprising:
forming the anti-fog film layer with a predetermined configuration and a proper thickness by cellulose acetate, wherein the lens substrate comprises the anti-fog film layer and a substrate layer;
forming the substrate layer by electroplating a component with a proper thickness that is transparent or not completely transparent; and
combining the anti-fog film layer and the substrate layer by an adhesive or non-adhesive to form the lens substrate.

2. The fabrication method of a lens substrate having an anti-fog film layer as claimed in claim 1, wherein the proper thickness of the anti-fog film layer of the lens substrate is substantially between 0.1 mm and 0.3 mm.

3. The fabrication method of a lens substrate having an anti-fog film layer as claimed in claim 1, wherein the adhesive combining the anti-fog film layer and the substrate layer of the lens substrate is implemented by an optically clear adhesive (OCA), ultraviolet (UV) curable adhesive, optically clear resin (OCR), or highly transparent adhesive.

4. The fabrication method of a lens substrate having an anti-fog film layer as claimed in claim 1, wherein the substrate layer of the lens substrate is implemented by polycarbonate (PC), triacetate cellulose (TAC) film, NYLON, CR-39 resin lens, polymethylmethacrylate (PMMA), polyethylene terephthalate (PET), or polypropylene (PP).

5. The fabrication method of a lens substrate having an anti-fog film layer as claimed in claim 1, wherein the substrate layer of the lens substrate is implemented with a spherical shape, a non-spherical shape, a planar shape, or a plate with a predetermined thickness and a configuration with a plurality of protrusions.

6. The fabrication method of a lens substrate having an anti-fog film layer as claimed in claim 1, wherein a surface of the substrate layer of the lens substrate is implemented to combine anti-scratch hard coating, UV400 protection, photochromic coating, or electroplating coatings such as multi-layer coating, water repellent coating, oleophobic coating, antireflection coating, anti-blue-ray coating, mirror coating, and color mirror coating.

7. The fabrication method of a lens substrate having an anti-fog film layer as claimed in claim 1, wherein the anti-fog film layer of the lens substrate is implemented with the predetermined configuration of a curved surface or non-curved surface according to a configuration of the substrate layer correspondingly.

8. A structure of a lens substrate having an anti-fog film layer, wherein the lens substrate is formed to achieve substantial thinning and keep expected anti-fog properties, the structure comprising:
an anti-fog film layer, formed with a predetermined configuration and a proper thickness by cellulose acetate; and
a substrate layer, formed by electroplating, combined on a lower surface of the anti-fog film layer by an adhesive or non-adhesive to form the lens substrate, wherein an overall thickness of the lens substrate is substantially thinned, and predetermined anti-fog properties of the lens substrate are kept after repeated cleaning or wiping after long terms.

9. The structure of a lens substrate having an anti-fog film layer as claimed in claim 8, wherein the proper thickness of the anti-fog film layer of the lens substrate is substantially between 0.1 mm and 0.3 mm.

10. The structure of a lens substrate having an anti-fog film layer as claimed in claim 8, wherein the adhesive combining the anti-fog film layer and the substrate layer of the lens substrate is implemented by an optically clear adhesive (OCA), ultraviolet (UV) curable adhesive, optically clear resin (OCR), or highly transparent adhesive.

11. The structure of a lens substrate having an anti-fog film layer as claimed in claim 8, wherein the substrate layer of the lens substrate is implemented by polycarbonate (PC), triacetate cellulose (TAC) film, NYLON, CR-39 resin lens, polymethylmethacrylate (PMMA), polyethylene terephthalate (PET), or polypropylene (PP).

12. The structure of a lens substrate having an anti-fog film layer as claimed in claim 8, wherein the substrate layer of the lens substrate is implemented by a component that is transparent or not completely transparent.

13. The structure of a lens substrate having an anti-fog film layer as claimed in claim 8, wherein the substrate layer of the lens substrate is implemented with a spherical shape, a non-spherical shape, a planar shape, or a plate with a predetermined thickness and a configuration with a plurality of protrusions.

14. The structure of a lens substrate having an anti-fog film layer as claimed in claim 8, wherein a surface of the substrate layer of the lens substrate is implemented to combine reinforced anti-scratch coating, UV400 protection, photochromic coating, or electroplating coatings of multi-coating, water repellent coating, oleophobic coating, antireflection coating, anti-blue-ray coating, mirror coating, and color mirror coating.

15. The structure of a lens substrate having an anti-fog film layer as claimed in claim 8, wherein the anti-fog film layer of the lens substrate is implemented with the predetermined configuration of a curved surface or non-curved surface according to a configuration of the substrate layer correspondingly.
